(19) United States
(12) Patent Application Publication
(10) Pub. No.: US 2021/0403294 A1
(43) Pub. Date: Dec. 30, 2021

(54) CEILING MOUNTED/POSITIONED (OVERHEAD) DELIVERY SYSTEM

(71) Applicant: S.W. Electronics & Manufacturing Corporation, Moorestown, NJ (US)

(72) Inventors: Richard SZCZEPKOWSKI, Moorestown, NJ (US); Joseph JESSON, Moorestown, NJ (US); Stephen SANGILLO, Moorestown, NJ (US); Richard LAFALCE, Moorestown, NJ (US)

(21) Appl. No.: 17/358,452

(22) Filed: Jun. 25, 2021

Related U.S. Application Data
(60) Provisional application No. 63/044,542, filed on Jun. 26, 2020.

(51) Int. Cl. B66C 13/08; A61L 2/10; B66F 3/22

(52) U.S. Cl. CPC B66C 13/085; A61L 2/10; A61L 2202/25; A61L 2202/11; A61L 2202/16; B66F 3/22

(57) ABSTRACT

A ceiling/overhead mounted delivery system includes a track configured to be mounted to a ceiling or overhead structure and extend in at least one of a first direction and a second orthogonal direction. A positioning tractor is attached to the track for motorized movement thereon. The delivery system may further include a delivery device, such as a selectively actuable germicidal light emitter. A third direction deployment module is coupled to the positioning tractor and the germicidal light emitter and is selectively adjustable to change a distance between the positioning tractor and the germicidal light emitter in a third direction. The third direction is substantially orthogonal to the first and second directions. A controller is configured to move the positioning tractor along the track, selectively operate the third direction deployment module to adjust the distance between the positioning tractor and the germicidal light emitter, and selectively actuate the germicidal light emitter.

### CROSS-REFERENCE TO RELATED APPLICATIONS

This application claims the benefit of U.S. Provisional Patent Application No. 63/044,542, filed on Jun. 26, 2020, entitled “Ceiling Mounted/Positioned (Overhead) UV Disinfectant Delivery System,” currently pending, the entire contents of which are incorporated by reference herein.

### BACKGROUND

Embodiments described herein relate generally to delivery systems, and more particularly, to delivery systems that are mounted to a ceiling or other overhead structure and are able to deliver devices, such as germicidal lamps, cameras, containers, or the like, to varying locations in a vehicle, facility, or the like.

In one embodiment, the overhead delivery system may be used to maneuver a germicidal light. Maximizing germicidal coverage in the shortest amount of time involves delivery of ultraviolet (UV) light at proper wavelength(s) as close as possible to potentially infected surfaces. Such surfaces typically have varying textures. The peaks and valleys formed in such surfaces can be, and often are, orders of magnitude larger than the germs that can infect humans. UV light is non-reflective, meaning the peaks and valleys of the textured surfaces can shadow the germs, reducing the coverage of fixed UV light fixture disinfecting methods. Conventional ceiling or wall mounted UV fixtures, as well as larger floor roving/moving fixtures, typically cannot get close enough to infected surfaces and require very long exposure times. Often it may take hours to achieve an acceptable germicidal result. These fixtures would also not be able to minimize shadowing affects that reduce coverage. Current movable cart style systems that move down a center aisle of a vehicle are also impeded by the various vertical poles and rails located throughout the vehicle. These carts systems also require an operator that needs to be shielded from the UV being emitted. Automation for this type of implementation would be significantly more expensive. These systems are also typically difficult to store in a vehicle since the systems involve a large cart that takes up floor space—necessitating external storage. The carts also need to be self-powered with large batteries that need to be recharged external to the vehicle.

It is therefore desired to provide a delivery system with one or more movable fixtures that can descend vertically for a variety of applications. For UV delivery systems specifically, it is desired to descend a movable fixture vertically to within a fraction of an inch of a targeted surface and address other issues described above.

### BRIEF SUMMARY

Briefly stated, one embodiment comprises a ceiling or overhead mounted delivery system that includes a track configured to be mounted to a ceiling or overhead structure and extend in at least one of a first direction and a second direction. The first direction is substantially orthogonal to the second direction. A positioning tractor is attached to the track for motorized movement thereon. The delivery system may further include a delivery device, such as a selectively actuable germicidal light emitter. A third direction deployment module is coupled to the positioning tractor and the germicidal light emitter and is selectively adjustable to change a distance between the positioning tractor and the germicidal light emitter in a third direction. The third direction is substantially orthogonal to the first and second directions. A controller is configured to move the positioning tractor along the track, to selectively operate the third direction deployment module to adjust the distance between the positioning tractor and the germicidal light emitter, and to selectively actuate the germicidal light emitter.

In one aspect, the track includes at least one set of electrical power rails. In another aspect, the at least one set of electrical power rails is connectable to one of an alternating current (AC) power supply or a battery. In another aspect, the positioning tractor includes at least one electrical contact roller configured for coupling to each respective electrical power rail of the set of electrical power rails.

In another aspect, the system further includes one or more sensors configured to record system information. The system information includes at least one of a position of the positioning tractor on the track, a position of the third direction deployment module, a tilt position of the germicidal light emitter, or a rotation position of the germicidal light emitter. In another aspect, the system further includes a communication module. The controller is configured to report the system information to an external device using the communication module.

In another aspect, the third direction deployment module is one of a scissor lift or a retractable cable.

In another aspect, the third direction deployment module includes a tilt and rotate module attached thereto and configured to selectively tilt and rotate the germicidal light emitter.

In another aspect, the germicidal light emitter is a lamp that emits light in one or more UVC wavelengths.

In another aspect, the positioning tractor includes at least one occupant sensor, and the controller is configured to shut off the germicidal emitter light based on a signal from the at least one occupant sensor.

In another aspect, the positioning tractor includes a drive motor and a drive roller operably connected to the drive motor and in contact with the track.

In another aspect, the controller is disposed within the positioning tractor.

In another aspect, the track includes a first portion extending in the first direction and a second portion extending in the second direction, and the track further includes a motorized turntable located at a junction between the first and second portions. The motorized turntable is configured to receive the positioning tractor and selectively rotate so as to switch the positioning tractor from the first portion to the second portion of the track.

In another aspect, the system further includes a communication module configured to receive commands from a remote device to change at least one of a position of the positioning tractor on the track, a position of the third direction deployment module, a tilt position of the germicidal light emitter, or a rotation position of the germicidal light emitter.

In another aspect, the controller is configured to store data related to a topography of a surface to be disinfected by the germicidal light emitter.

Another embodiment comprises a vehicle including a ceiling, at least one surface to be disinfected located vertically beneath the ceiling, a power supply, a track attached to the ceiling, and a positioning tractor attached to the track for motorized movement thereon. The positioning tractor receives power from the power supply. The vehicle further includes a selectively actuable germicidal light emitter and a vertical deployment module coupled to the positioning tractor and the germicidal light emitter and being selectively adjustable to change a vertical distance between the positioning tractor and the germicidal light emitter. A controller is configured to move the positioning tractor along the track, to selectively operate the vertical deployment module to adjust the vertical distance between the positioning tractor and the germicidal light emitter, and to selectively actuate the germicidal light emitter to disinfect the at least one surface.

In one aspect, the power supply is a vehicle battery.

In another aspect, the at least one surface to be disinfected includes at least one vehicle seat.

In another aspect, the controller is configured to store data related to vertical distances of points on the at least one surface from the positioning tractor.

### BRIEF DESCRIPTION OF THE SEVERAL VIEWS OF THE DRAWINGS

The following detailed description of preferred embodiments will be better understood when read in conjunction with the appended drawings. For the purpose of illustration, there are shown in the drawings embodiments which are presently preferred. It should be understood, however, that the invention is not limited to the precise arrangements and instrumentalities shown.

In the drawings:

FIG. 1 is a side perspective view of a UV delivery system in accordance with a first embodiment of the present invention;

FIG. 2 is a top perspective view of a track and positioning tractor of the UV delivery system of FIG. 1;

FIG. 3 is a side elevational cross-sectional view of the track and positioning tractor of FIG. 2;

FIG. 4 is a front side elevational partial cross-sectional view of the track and positioning tractor of FIG. 2;

FIG. 5 is a top perspective view of the positioning tractor of FIG. 2;

FIG. 6 is a top perspective view of a track in accordance with a second embodiment of the present invention;

FIG. 7 is a top perspective view of a track in accordance with a third embodiment of the present invention;

FIG. 8 is a front perspective view of a drop ceiling clip and threaded mounting coupler for mounting the track shown in FIG. 1;

FIG. 9 is a bottom perspective view of the positioning tractor of FIG. 2;

FIG. 10 is an exploded view of the positioning tractor of FIG. 2;

FIG. 11 is a top perspective view of a z-axis deployment module of the UV delivery system of FIG. 1 in a closed configuration;

FIG. 12 is a top perspective view of the z-axis deployment module of FIG. 11 in a fully expanded configuration;

FIG. 13 is a side perspective view of a tilt and rotate module of the UV delivery system of FIG. 1;

FIG. 14 shows schematic views of the positioning tractor of FIG. 2 with a germicidal light emitter directly attached thereto in accordance with an alternative embodiment;

FIG. 15 is a screenshot of a web interface for review and control of the UV delivery system of FIG. 1; and

FIG. 16 is a schematic block diagram of a general overhead delivery system in accordance with additional embodiments of the present invention.

### DETAILED DESCRIPTION

Certain terminology is used in the following description for convenience only and is not limiting. The words “right”, “left”, “lower”, and “upper” designate directions in the drawings to which reference is made. The words “inwardly” and “outwardly” refer to directions toward and away from, respectively, the geometric center of the device and designated parts thereof. The terminology includes the above-listed words, derivatives thereof, and words of similar import. Additionally, the words “a” and “an”, as used in the claims and in the corresponding portions of the specification, mean “at least one.”

It should also be understood that the terms “about,” “approximately,” “generally,” “substantially” and like terms, used herein when referring to a dimension or characteristic of a component, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

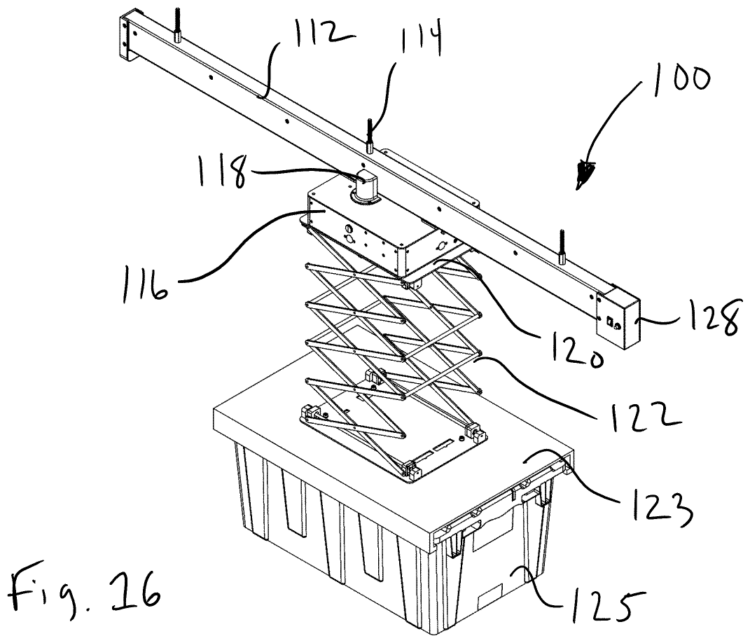

Embodiments described herein are directed to a ceiling positioned/mounted (overhead) delivery systems to support mechanical deployment, movement, positioning, and orientation of various delivery devices to various locations within a vehicle, facility, or the like in an efficient, safe, and reliable manner. This application in particular describes one example of such a delivery system for moving a UV emitter fixture for disinfectant purposes. While much of the description herein is directed at such an embodiment, it is to be understood that the delivery system can have other, unrelated purposes as well, which are also described herein and shown, for example, in FIG. 16, which is discussed later.

In relation to an overhead disinfectant system, one of the goals of the system is to achieve maximum disinfection of surfaces. Such surfaces may be located within vehicles (such as rail cars, busses, or the like) as well as within facilities (such as nursing homes, elevators, subways, factories, or the like)—virtually any public place where infections can be passed from one person to the next due to surface contamination/exposure. The delivery system may facilitate the movement and positioning of UV disinfecting emitters/fixtures/lamps to get as close as possible to surfaces that could be contaminated. Such surfaces may be horizontal, vertical, textured, combinations thereof, or the like. Such surfaces may be located anywhere between a floor and ceiling. Ceiling mounted/positioned embodiments are not affected by seats, furniture, work benches, beds, or the like and may be deployed from above following an installed guidance track. The UV disinfecting emitters/fixtures/lamps preferably may get within an inch or less of surfaces to be disinfected, significantly maximizing the UV intensity that reaches the surface, minimizing shadowing, and shortening the time necessary to accomplish the disinfection (e.g., minutes instead of hours). The light may also kill pathogens that are airborne in the vicinity of the targeted surfaces.

Conventional fixed mount or floor-located or traversing systems may be affected by bumping into furniture and other objects as opposed to being deployed and moving from above where there are no impediments. For systems affected by impediments, disinfecting time is significantly increased due to the inability to get the UV emitter lamps close enough to the targeted surface to be disinfected. Various embodiments of the ceiling positioned/mounted (overhead) UV delivery system will include a mechanism to raise and lower attached UV emitter fixtures into orientations necessary to get as close as possible to surfaces that may be horizontal, vertical, shadowed, or the like. For example, it is expected that a rail car, subway car, or transit bus could be disinfected in ten to twelve minutes. This is significantly faster and with much higher coverage than could be achieved using fixtures mounted in place that could take several hours regardless of how many fixtures are utilized. The ceiling mounted delivery system also tends to be consistently out of the way of the public or a driver and does not impede vehicle or building floor space. Since the system may be integrated into the vehicle/facility, the system also can be powered directly from the vehicle/facility or recharged by the vehicle or facility power, depending on the embodiment. For example, rail vehicles are continuously powered and could easily power an installed UV delivery system and associated UV emitters/fixtures. Busses could utilize a relatively small rechargeable battery mounted in the positioning tractor of the system as operation time for the ceiling mounted system is quite short at an approximate ten to twelve minutes. The vehicle charging system could be utilized to recharge batteries associated with the installed ceiling mounted UV delivery system and associated UV emitter fixtures.

A computer and control subsystem may be utilized as part of the system to support various programmable features. Examples include the following:

(1) Utilization of pre-programmed disinfecting map routines targeting a specific facility or vehicular space. The routine may support all positioning (horizontal, vertical, angular, rotational, and the like) as necessary to achieve best coverage.
(2) The ability to run such routines manually or on a preset schedule.
(3) The ability to use the built-in system sensors to facilitate initial programming and/or provide adaptive capability for any changes in the targeted space such as movement of furniture, tables, freight, personal possessions, or other such items that can affect the UV disinfecting operation.

Pre-programmed routines support easier/repeatable documentation of the disinfecting process, leaving no ambiguity as to coverage. For example, this can help with certification/approval of disinfecting methods for peace-of-mind and/or legal considerations. Competitive roving systems, in addition to being impeded by furniture and other items on the floor, are much like a roving room or pool vacuum and may not get to every spot to be disinfected in a repeatable manner. Programming routines are also accurate and could be updated via wired or wireless WAN/LAN techniques. This supports ease of updates and or retrieving operational information, maintenance status, system health, and the time/date/history of running the disinfecting process.

Various embodiments described herein include a ceiling mounted/positioned (overhead) delivery system that can place UV light sources/fixtures very close to most surfaces likely to be infected, such as in rail cars, busses, theaters, nursing homes, elevators, and other like places that have high public access.

Various embodiments described herein may allow one or more UV light sources to be deployed in the “x”, “y”, and “z” axes and may also include fixture mounts that have rotational or angular capability. The rotational/angular capability may allow more complete minimization of shadowing effects due to both the environment and texture of potentially infected surfaces. Fixtures may contain one or more UV emitters as deemed necessary for the germicidal application.

Various embodiments described herein may also support using any/all current or newly available UV fixture technologies and UV emitter lamp or multi-lamp array types.

One or more embodiments of the delivery system described herein may support multiple installations within a facility or vehicle. For example, multiple delivery systems may be installed in a transit bus. This would eliminate moving the equipment around various vertical poles that are held by riders when standing. One embodiment might encompass three separate systems; one to traverse the center aisle, one to traverse the left side seats, and one to traverse the right side seats. A lower cost embodiment may use a single routed track and UV positioning tractor. This implementation may use a continuous track routed to cover the complete targeted facility or vehicle. However, this implementation may necessitate a longer disinfecting time than if multiple tracks and UV light fixture positioning tractors are used. Embodiments described herein may be utilized to accomplish optimum UV light fixture emitter deployment in optimum places to maximize germicidal coverage.

In addition to the ceiling mounted tracks, various embodiments may include structural components that could be deployed/monitored vertically to get as close as possible to targeted surfaces. Implementations could include deployment via cable that could be reeled in the upper mechanism to facilitate longer vertical deployments with minimal structure that would need to be stored when the system is not in use, saving overhead space and possibly cost. Depending on factors such as the required vertical deployment, some embodiments may use a mechanical folding structure or other such vertical positioning approach to accomplish the vertical deployment. In either case, the movable tractor preferably supports the motor drive interface, control signals and mechanical mounting to add the vertical and/or tilt/rotate deployment mechanism(s) as a bolt-on module(s) to the positioning tractor.

The delivery system may further include support for both position and UV intensity sensors to support position deployment control as well as the logging of disinfecting results.

Various embodiments may include computer control to support both operation and communication of disinfecting results via Wi-Fi, cellular, LPWAN, or other such wired or wireless techniques.

Various embodiments may include automated and programmable profiles for various vehicles, facilities, or the like. Thus, fully automated operation, including scheduled disinfecting could be supported. Programmable and/or scheduled deployment also allows for operation without an operator being present in the space to be disinfected, which reduces the potential for operator exposure to harmful UV wavelengths. Such implementations may further include motion/occupant detection to prevent inadvertent system operation and exposure to operators or the public.

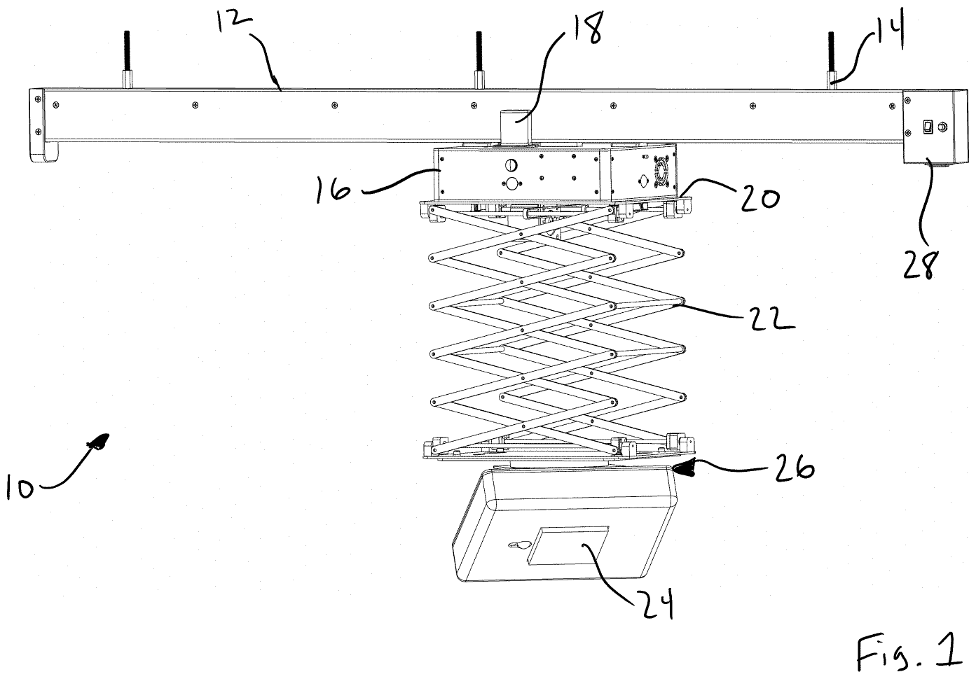

FIG. 1 shows an example embodiment of a UV delivery system 10. The UV delivery system 10 may include a track 12 attached to a ceiling or other overhead structure (not shown) via track mounting hardware 14. The track 12 supports a positioning tractor 16 operated to move along the track 12 via a drive system 18. An underside of the positioning tractor 16 may include an attachment plate 20 supporting a z-axis deployment module 22 for raising and lowering a germicidal light emitter 24, such as a UV light. The germicidal light emitter 24 may be oriented using a tilt and rotate module 26. Power to the UV delivery system 10 can be at least partially provided by a power input block 28 located at an end of the track.

An upper support track structure is one important feature that allows the delivery system to provide complete area coverage of the targeted space to be disinfected. This is true for any covered space whether within a vehicle or building. The track can position the UV disinfecting emitters/fixtures/lamps wherever needed within the floor area of a vehicle or facility room. This includes being above any furniture, shelves, seats, beds, and the like. Examples of vertical positioning features described in more detail below may be used to place the UV disinfecting emitters/fixtures/lamps as close as possible to the targeted surface to be disinfected regardless of surface orientation.

Embodiments of the track structure can vary widely as a function of the desired application. Typically, the track may be mounted to a ceiling or other overhead structure of a space to be disinfected. Some applications involve totally flat mounting surfaces, such as building ceilings, while others could be curved, as is often the design utilized within vehicle interiors. Tracks may have sensors that support full or partial automation to accomplish the UV disinfecting process in an automated manner with no operator present. These sensors will feed control electronics described in further detail below.

Embodiments may include one or more separate tracks as required to, for example, achieve coverage around obstacles and/or to facilitate accomplishing the disinfecting process in the fastest possible time. For example, a vehicle system for a bus may include three separate track systems that traverse from the front of the bus to the back or vice versa. Such an implementation provides the simplest control without needing to run the track in the direction of the vehicle width to cover left seats, center aisle, and right seats utilizing separate passes from one end of the bus to the other. Three separate track systems can simultaneously cover the length of a bus in one pass in the simplest possible manner and in the shortest possible time. Other embodiments may use a single fixture or fixture array that can travel on a track up and down the vehicle or transition between tracks in order to accomplish disinfection of the left seats, center aisle, and right seats via successive passes traversing the length of the vehicle. The decision to use one or the other may be a cost versus disinfecting time tradeoff, for example.

The ceiling mounted/positioned (overhead) structure and associated components can support the sensors needed for basic operations of position/limit sensing as well as pre-programmed operation, adaptive control and positioning, and updates to pre-programmed operational maps and routines.

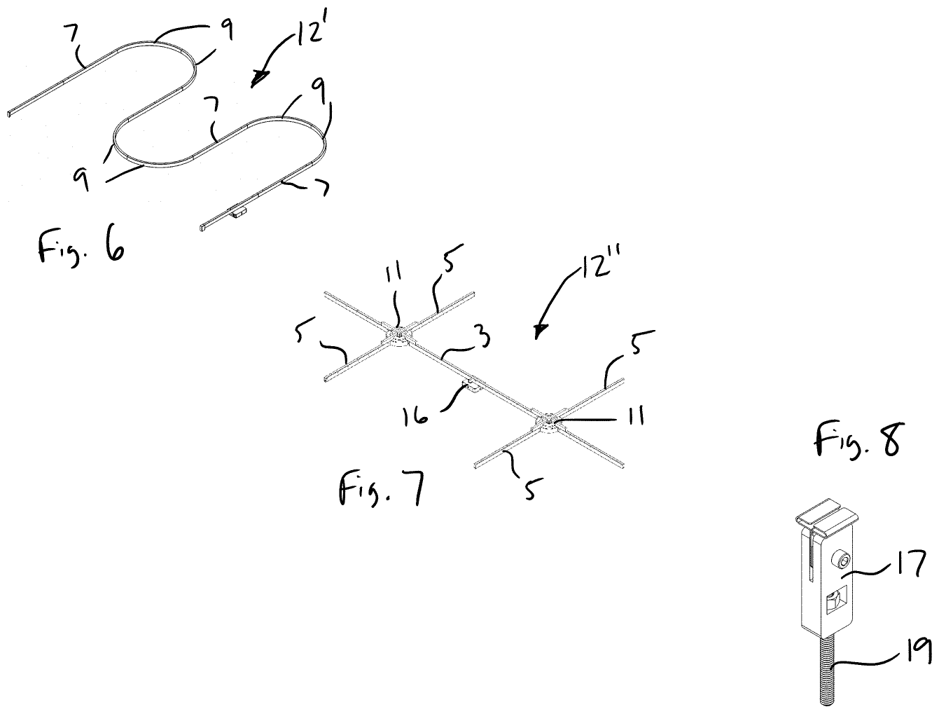

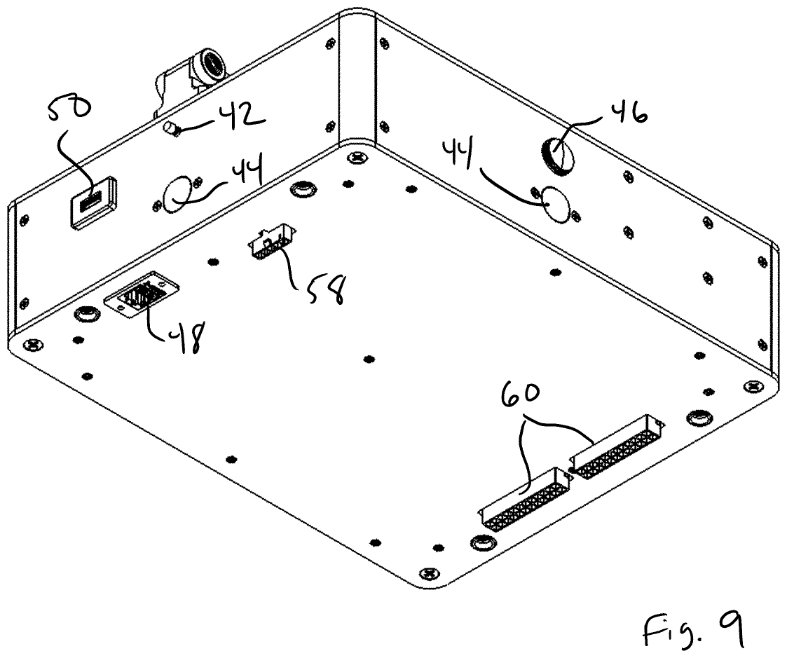

The delivery system preferably includes a powered track supporting a tractor that can travel along the track. UV fixtures of various types may be attached to the tractor and moved about the space to be disinfected. Track layout may be accomplished via connecting together various lengths of track in straight and/or curved sections, although other methods for providing a track, such as a single piece track or the like, may be utilized as well. For example, FIG. 6 illustrates a track 12′ having a plurality of straight sections 7 connected by a series of curved sections 9 to form an overall curved “M” shape. Track switching may also be accomplished utilizing a turntable that accepts the tractor, rotates, and connects to another track segment at 90 degrees or other possible angles of rotation. For example, FIG. 7 shows a track 12″ having a generally straight main section 3 and a plurality of branches 5 extending at approximately 90 degrees with respect to the main section 3. At each of the junctions with the branches 5, a turntable 11 may be provided as described above for receiving and diverting the positioning tractor 16. The turntables 11 may be powered by the track 12, the positioning tractor 16, when connected, or the like for facilitating rotational movement. Sections of any track 12, 12′, 12″ can vary in length to suit the desired application.

The track may include the following features and advantages:

(1) Easily attaches to ceilings including drop ceilings via custom attachment blocks.
(2) Easily supports the weight of the tractor and attached UV fixtures or other payload.
(3) Provides a combination roller surface and track attachment to facilitate safe operation of the tractor. No risk of detachment or separation of the tractor from the track.
(4) Insulated copper power strips run the full length of each track section in order to provide electrical power to the tractor.
(5) Provides sufficient power capability to operate the positioning tractor along with anticipated UV attached fixtures or arrays of fixtures.
(6) Insulated copper power strips are recessed and insulated so as to prevent inadvertent human contact either directly or from tools when doing maintenance.
(7) Track sections are designed to minimize any dust/debris buildup from the environment.
(8) Non-powered overhead tracks are also possible; however, battery powered positioning tractors would be utilized for such applications.

Power may be passed from the track to the tractor via compliant rolling contacts for each power rail and frame ground. Track sections are preferably seamlessly pluggable with one another.

In addition to straight and curved track sections, directional change or track switching can be accomplished via a circular turntable or the like. The turntable may include a section of powered track that can be rotated. The tractor first drives onto the turntable and stops. The turntable can then be rotated manually or automatically to allow connection to another section of track. Specific requirements of the targeted space to be disinfected can determine whether curved track sections and/or turntable(s) are the optimal structures to change tractor direction.

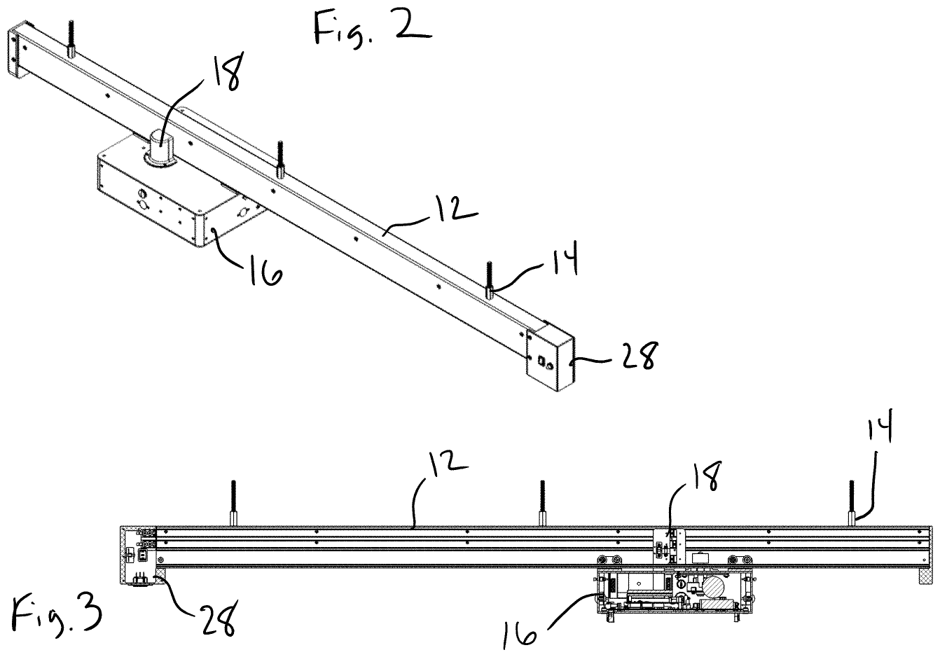

An example track 12 is shown in FIGS. 1-3. Track mounting hardware 14 may be used to attach the track 12 to a ceiling or other overhead structure. The track mounting hardware 14 in the example of FIGS. 1-4 is shown as a series of threaded mounting studs spaced along a top of the track 12 and each of which is configured to receive a threaded mounting coupler 19 (FIG. 8) FIG. 8 shows one example of a drop ceiling clip 17 that receives and suspends a threaded mounting coupler 19 that can be received by the track mounting hardware 14 of the track 12. However, other types of mounting hardware can be used depending on the application. For example, the track mounting hardware 14 can additionally or alternatively be in the form of mounting brackets or other types of mechanical fasteners. The track mounting hardware 14 may, in some instances, allow for quick attachment or detachment to the ceiling or overhead structure, as necessary, or may facilitate a more permanent installation. In still other embodiments, the track 12 may be attached by other methods, such as by adhesives, welding, or the like.

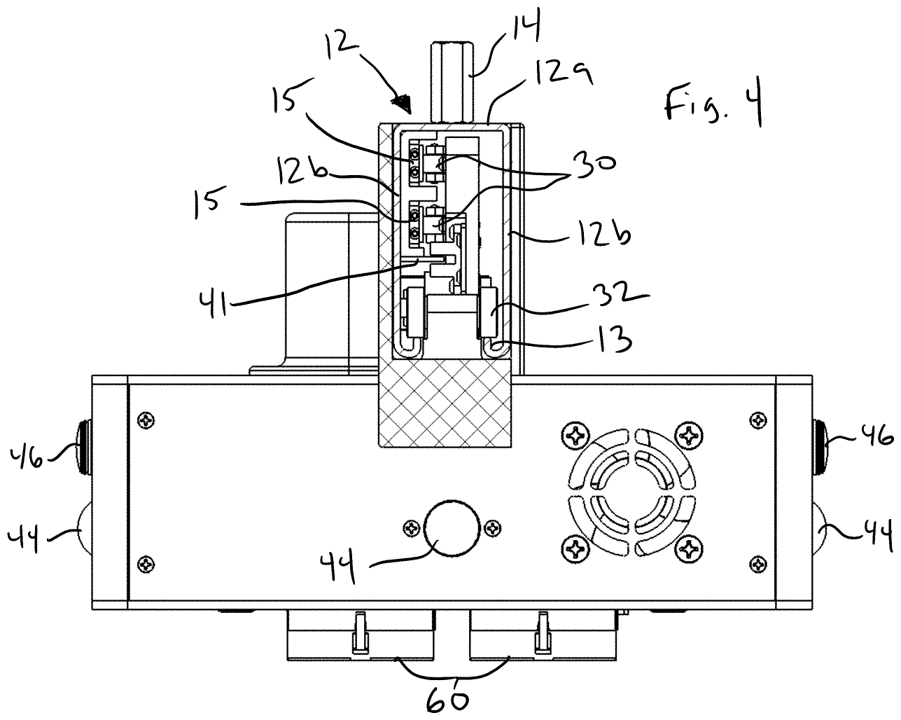

As shown in FIGS. 3 and 4, the rail 12 may be hollow, formed by a top plate 12a connected with opposing sidewalls 12b extending generally vertically therefrom. Each sidewall 12b may form a lip 13, as will be described in more detail below. A plurality of electrical rails 15 may be disposed within the track 12 and run along at least a portion of a longitudinal extent thereof. The electrical rails 15 preferably receive power via the power input block 28 at an end of the track 12, which can be connected to a battery, mains power outlet, or like power supply as available in the space to be disinfected. The electrical rails 15 are preferably mounted within the hollow space of the track 12 in order to prevent unintentional contact and reduce the risk of electrical shock, although in some embodiments such electrical rails 15 may be located on other portions of the track 12. The electrical rails 15 can be used to supply power to the positioning tractor 16, as described in further detail below. However, other methods of power delivery using the track 12 may be used as well.

Control and operation of the delivery system is preferably managed and facilitated by a positioning tractor and associated components. Potential aspects of the positioning tractor include:

(1) Ability to traverse, position, and operate anywhere along the overhead track.
(2) The positioning tractor may be the primary system component onto which all additional operational system positioning and sensing components can be attached.
(3) Various UV emitter types may be attached directly to the positioning tractor via an interface plate specific for each UV emitter product.
(4) All power, operational control, motor drive/control, position sensing, occupant sensing, and remote configuration/control may be accomplished by the positioning tractor.
(5) Multiple electrical contact rollers of the positioning tractor can prevent inadvertent electrical dropouts should any contact imperfection occur for any reason. The rollers especially facilitate the transfer of the tractor from one track section to the next or to a turntable that would be used for track switching.
(6) Electrical contact may be further facilitated by an Uninterruptible Power Supply (UPS) on a main printed circuit board. The UPS electronics may continue to support power to microprocessor controllers in the event of any power glitches or short-term loss of primary power.
(7) A programmable relay power switch may be provided to allow software control of power to the attached UV fixture(s). An example would be if someone enters a room while disinfection is in process or the system detects an internal failure. The system may automatically remove power to the UV emitter fixture(s) until a safe condition is detected.
(8) Occupant sensing by either the positioning tractor or an attached UV emitter fixture may be supported along with associated remedial action.
(9) Motor drive circuitry may support either stepped or pulse width modulated (PWM) speed control of the positioning tractor and any bolt-on positioning actuators.
(10) A controller may provide direct support for four motors (e.g., a tractor drive motor and three others to be used for vertical deployment, angular, and rotational movement of the attached UV fixture). Additional motors or features could be added via various versions of the main controller printed circuit board in the positioning tractor.
(11) Control for the additional motors may pass through an interface connector at the base of the positioning tractor.
(12) The mechanics for the additional vertical, angular, or rotational features may be optional as bolt-ons to the positioning tractor.

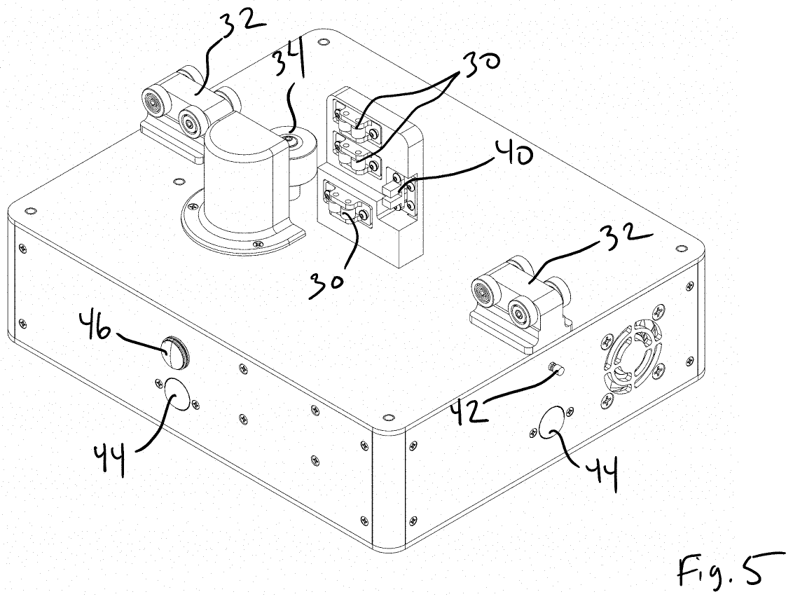

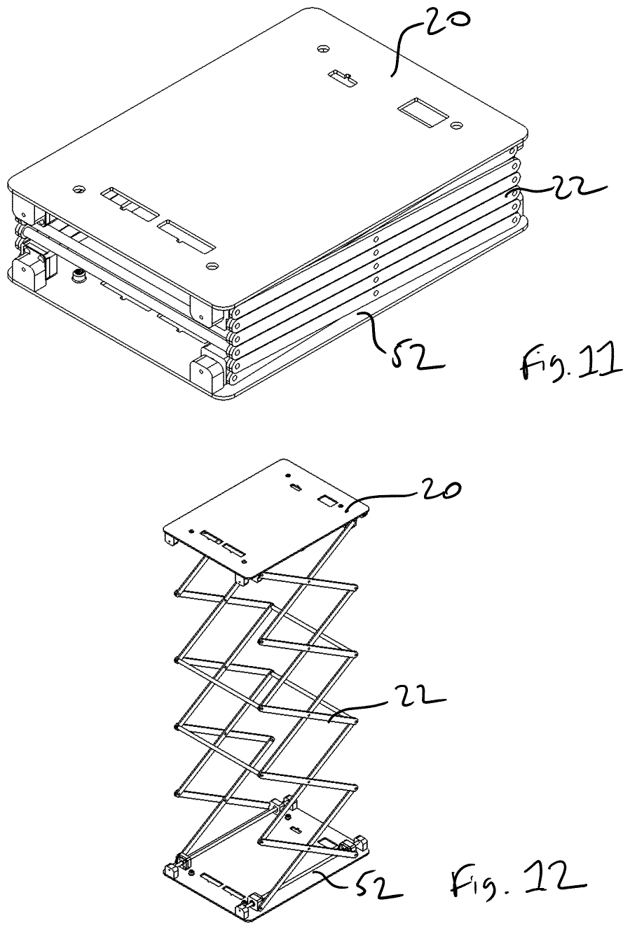

Various aspects of an example positioning tractor 16 are shown in FIGS. 4-5 and 9-10. The positioning tractor 16 may receive power from the electrical rails 15 of the track 12 using contact rollers 30 that are preferably tensioned into contact with the electrical rails 15 when the positioning tractor 16 is mounted on the track 12. The contact rollers 30 may be received within the hollow portion of the track 12 for safety purposes. While contact rollers 30 are shown as the providing electrical power to the positioning tractor 16 are shown in this example, other methods of supplying power may be used as well, such as a flexible cord, a separate power supply, or the like. The positioning tractor 16 may have an internal DC power supply 45, which may be configured to rectify AC power received over the electrical rails 15, regulate voltage from the externally supplied power, provide back-up power, or the like. Alternatively, the DC power supply 45 may be the primary power source for the positioning tractor 16 in some circumstances.

To retain the positioning tractor 16 on the track 12, a plurality of guide rollers 32 are positioned and spaced apart on top of the positioning tractor 16 and are configured to be received on the lips 13 of the track 12 formed by the sidewalls 12b. However, other methods of suspending the positioning tractor 16 on the track 12 may be used as well, including slidable, rolling, and/or other attachment types.

The positioning tractor 16 may be moved along the track by engagement of a drive roller 34 with a surface of the track 12 and cooperating with a tension roller 35. The drive roller 34 may be driven by a drive motor 36 housed within the positioning tractor 16. However, other methods of propelling the positioning tractor 16 along the track 12 can be used as well. For example, a drive gear (not shown) operated by the drive motor 36 may engage with a row of teeth (not shown) on the track 12. In other embodiments, the track 12 may provide a motor that moves portions of the track 12 to initiate relative motion of the positioning tractor 16.

Operation of the drive motor 36 may be governed by a controller 38 disposed within the positioning tractor 16 and on a main control printed circuit board 39. The controller 38 may be any type of controller, such as a microcontroller, microprocessor, multiple processors, or the like. The controller 38 preferably includes or is operatively coupled to a memory that may store code or software for carrying out the processes described herein, or for carrying out other operations of the UV delivery system 10 and may store any captured data for later transfer to remote or external devices. It should be further appreciated that controller 38 is shown in this example as a single component, but may include a plurality of individual devices, with control functions divided among the individual devices.

As will be described in more detail below, the controller 38 preferably is able to determine the position of the positioning tractor 16 on the track 12 to allow for appropriate adjustments of the z-axis deployment module 22. For example, the positioning tractor 16 may include a track position sensor 40, which may be in the form of an optical reader that cooperates with an encoder bar 41 running along the track 12. The encoder bar 41 may have markings, openings, notches, or the like that can be detected by the track position sensor 40 and interpreted by the controller 38 to determine the position of the positioning tractor 16 on the track 12. Other methods for determining position can be used as well, such as switches, capacitive or inductive sensing, motor revolution counting, or the like. In addition, the positioning tractor 16 may include track end sensors 42 indicating the start or end point of the track 12, junctions, or the like. In this example, the track end sensors 42 are shown as physical switches that are triggered via abutment with a structure of the track 12 such as an end plate, like the power input block 28. However, other methods for detecting that the positioning tractor 16 has reached the end of the track 12, including using the track position sensor 40 itself, can be used as well. Triggering of one of the track end sensors 42 can cut power to the drive motor 36 and/or cause the controller 38 to take other actions.

The positioning tractor 16 is preferably also equipped with one or more occupancy sensors 44 configured for detecting the presence of individuals in the vicinity of the UV delivery system 10. When the germicidal light emitter 24 emits light in the UV range, for example, such light is harmful to humans. The controller 38 may receive data from one or more of the occupancy sensors 44 indicative of a human presence and, for example, cut power to the germicidal light emitter 24, emit a warning, or the like. The occupancy sensor 44 may be a passive infrared (PIR) type sensor, a temperature sensor, an ultrasonic sensor, or the like.

The positioning tractor 16 may further include one or more indicator lights 46, which can be used to provide visual alerts, such as for warnings or signaling abnormal conditions, or may provide visual indication of an operating status. The indicator lights 46 may each include one or more LEDs and may change colors. Other on-board indicators can be provided alternatively or in addition to the indicator lights 46, including display screens, speakers or other audio outputs, combinations thereof, and the like.

The positioning tractor 16 may include one or more communication modules for sending and/or receiving data, instructions, commands, or the like. In the example shown in the drawings, the positioning tractor 16 includes an Ethernet port 48 and a USB port 50. The positioning tractor 16 may alternatively or additionally include a wireless communication module configured for Wi-Fi, cellular, LPWAN, or other like wireless protocols. Other wired protocols may be used as well. As will be described in further detail below, the communication module(s) may receive commands or data from a remote or external device (not shown), such as for firmware updates, map updates, requesting data or status reports, changing positions on the track 12, changing the positioning of the germicidal light emitter 24, and the like. Similarly, the communication module(s) may be used for reporting stored data to a remote or external device (not shown), as is explained further below.

One aspect to achieving maximum coverage and disinfecting speed of the delivery system is the ability to traverse any space to be disinfected in horizontal and vertical directions throughout the targeted environment. The overall system may be used to deploy one or more UV disinfecting emitters/fixtures/lamps as close as possible to targeted surfaces to be disinfected.

As in all volumetric positioning systems, a position within the volumetric space can be located by the associated X, Y, and Z coordinates. In addition, the speed at which the associated UV emitters/fixtures/lamps can reach any targeted surface, traverse the surface, and then move to another surface to be disinfected is controlled by the velocity or drive capability (V) of the positioning system. The design of the UV delivery system and associated emitters/fixtures can vary based on the various sizes and/or shapes of the targeted environment, whether a building space, vehicular space, factory, public area, or the like. An example may be whether the vertical portion of the system (Z) is deployed via a cable-based implementation or a movable mechanical arm based system. Whether cable deployed or mechanical arm deployed, the delivery system uses the ceiling mounted/positioned track and associated positioning tractor for traversing the area (X and Y) of the targeted space to be disinfected. The horizontal and vertical deployment operation may involve motorized drive actuator(s) to control the speed of deployment in the X, Y, and Z directions. Deployment in the Z direction may be via a mechanical bolt-on module to the positioning tractor. The rate of motion (V) may be determined based on the required deployment speed, disinfecting speed, method of power (direct AC or battery), cost, component availability, and any other such factors. Speed control may be via stepped or PWM methods, both of which may be supported by the main control board mounted in the positioning tractor.

Control of the UV delivery system may be via the main control board mounted in the positioning tractor. Configuration and programming can either be accomplished by directly attaching a computer or remotely via Wi-Fi or other wireless networking capability. Software/firmware on the main control board can manage the various combination of position sensors (mechanical, optical, proximity) and optional UV exposure sensors via a remote portal. Exposure sensors may support logging as proof of coverage and/or for setting/fine tuning programmable exposure features. The ability to have custom programmable maps/profiles for various targeted spaces is part of the architectural system design to be implemented as required.

A number of embodiments of the UV delivery system may be permanent or semi-permanent installations. In such systems, the power subsystem is likely connected to the building or vehicle power. For building installations, no battery powered auxiliary system would be needed unless requirements include the ability to operate when primary building power fails due to some outage or other similar situation. A rechargeable battery subsystem can be added as an attachment to the positioning tractor or can be installed where primary power feeds the powered overhead track.

Vehicular embodiments of the delivery system will likely have varying power subsystem implementations. Electrically propelled rail cars are typically continuously powered. In such situations, the power subsystem for the disinfecting system could be continuously powered similar to what would be used in building applications. Special situations could possibly necessitate supporting battery power. Associated battery bolt-ons could be added to the positioning tractor or to the primary power source feeding the powered overhead track. A battery subsystem could include the ability to monitor the battery state of charge either locally or remotely. This is via the communications capability of the computerized control and communication system of the main control board mounted in the positioning tractor.

Transit and school busses are only powered when the engine is running. Depending on specific requirements, an auxiliary power system may be required. It is anticipated that a power converter could be used to transform the 24 VDC vehicle power to a standard 115 VAC that may be used to power the UV delivery system. If needed, supplemental battery support could be added. Since the operating time is preferably quite short (preferably approximately ten to twelve minutes), the system could be powered/recharged by the vehicle battery when the positioning tractor is docked/parked. Once disinfected, there may not be a need to disinfect again until the bus runs its intended route, which will present the opportunity to recharge any lost battery capacity. Some applications may require or prefer a larger auxiliary battery. In such cases an auxiliary rechargeable battery subsystem could be provided. The vehicle charging system could also recharge the auxiliary battery in addition to the primary vehicle battery while the bus is running its intended route. The size of the auxiliary battery subsystem is expected to be small due to the preferred short run time of the UV delivery system needed to accomplish the vehicle disinfecting process or cycle. Utilizing the vehicle battery and associated power conversion/charging system eliminates the need for large rechargeable batteries and the systems that would be needed to recharge them external to the vehicle. As above in the rail car example, local and/or remote sensing of the battery state of charge could be supported.

The UV delivery system may be designed to use commercial, off-the-shelf UV lamps, lamp arrays, and associated fixtures as deemed appropriate for each application environment. Thus, the UV delivery system is preferably designed to be flexible based on the lamp or fixture technology required to best accomplish the disinfecting task for each targeted use/application. Various lamp/fixture embodiments may utilize a similar mounting plate. This supports keeping the delivery tracked system with one or more fixture mounting heads as common as possible among the varying lamp/fixture UV emitter options. The UV delivery system preferably can work with any/all possible UV disinfecting lamp technologies. Examples include but are not limited to: 254 nm UVC lamp(s); 222 nm far-UVC lamp(s); pulsed UV Xenon lamp(s); UVC LED arrays, combinations thereof, or the like.

The selection of lamp type(s) may vary depending on the size and orientation of targeted surfaces to be disinfected, shadowed effects on the various surfaces, facility or vehicle size and shape, objects (such as furniture or obstructions within the targeted space), etc. Other significant factors include the intensity required for the disinfecting process and the time required to complete the process. Systems with multiple lamps, lamp arrays, and fixtures may be used to achieve coverage in a faster manner or to facilitate a higher coverage.

A lamp/fixture mounting head can vary in size based on the intended application. An example embodiment for a transit bus would likely utilize one or more mounting head(s) each of which is roughly 2 feet×6 inches to achieve disinfecting coverage in an approximate ten to twelve minute cycle time for the process.

The UV disinfecting process would be run when there are no occupants in the targeted area within a building or vehicle because the emission from the most effective UV emitter lamp types is harmful to humans. The UV delivery system design may include sensors to ensure that there are no occupants.

Both horizontal and vertical track positioning systems may utilize sensors to define endpoints of travel, e.g., the end of the physical horizontal track or the vertical height from which to operate the UV emitter lamps. The combination of horizontal and vertical controlled positioning can allow the UV emitter lamps to be placed as close as possible to the surface to be disinfected and in the proper orientation to achieve maximum germicidal effect.

Once deployed to an optimum position for disinfecting a specific surface within the facility or vehicle, the UV delivery system may have the ability to further redirect the UV emitter lamp illumination at various angles for best direct illumination, minimization of shadowing, and/or minimization of the effects of surface texture. This can be manually controlled for setup/configuration, pre-programmed and/or adaptive. The surface texture can create a micro version of shadowing effects as typical germs are nearly always smaller than the crevices of textured surfaces. Varying the angle of incidence of the applied UV light is a way to mitigate this issue, greatly improving germicidal efficiency. Varying the light angle may be accomplished by varying the horizontal angle, vertical angle, or via rotation of the emitter lamp(s). These micro positioning operations may be optional bolt-on features to the positioning tractor and UV light fixture emitter operation. Tilt and rotate features may be integrated into a single bolt-on module that is separate from the vertical deployment module.

Another optional feature can be provided to sense the intensity and duration of the UV emitted light. An example embodiment of this feature may include mounting the sensor on a light fixture carrier where it can be deployed at a set distance that would mimic the distance from the surface being disinfected. The sensor could be used for both calibration/programming of an automated germicidal process or for logging purposes as may be needed by a customer.

The UV delivery system is a mechanized disinfecting system requiring computer control of all operations. There is no operator to move the UV light fixture carrier throughout the facility or vehicle space to be disinfected. The positioning tractor along with attached UV fixture emitter is typically powered from the overhead track. Primary power is provided to the track directly from building or vehicle power. Some embodiments may utilize a power conditioner, battery, or UPS as needed for the application. Battery power can be applied either at the primary power input to the track or can be attached to the positioning tractor as a bolt-on option. The system can be implemented with or without the overhead track being powered. If not powered, the positioning tractor will support a battery to power the drive system as well as powering the attached UV emitter fixture(s). The battery pack most likely is recharged directly from building or vehicle power. Recharging of the battery in the positioning tractor is provided at the docking position where stored prior to use, or the battery pack could be removed for recharging. Initial embodiments will have the track mounted at the top of the facility/vehicle space. The overhead track has built in markers that could be detected by electronics in the positioning tractor. This allows the positioning tractor and associated UV emitter fixture(s) to be positioned anywhere along the track for best disinfecting operation. Thus, the system can be positioned to the start or end of a track, or anywhere along the track. Power, control, actuator, positioning, motor drive, sensing, communication, and logging functions are incorporated into the positioning tractor. In addition to traveling along a track, the system supports positioning the UV fixture(s) as close as possible to surfaces to be disinfected. This is accomplished by utilizing vertical deployment and/or tilt/rotate bolt-on modules to the positioning tractor.

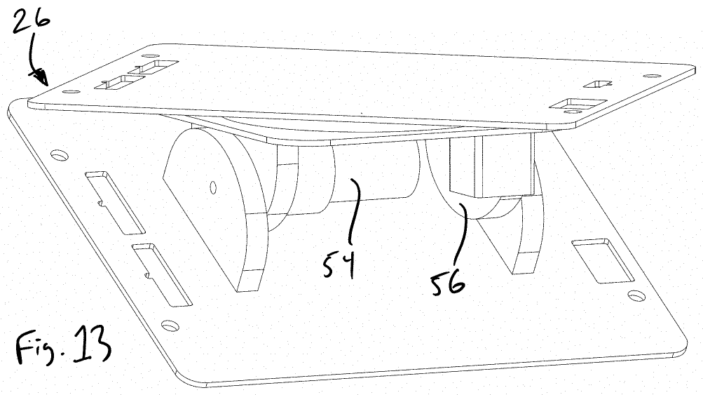

FIGS. 11 and 12 show one example of a z-axis deployment module 22 for use with the positioning tractor 16. In this example, the z-axis deployment module 22 is a scissor lift, but other types of deployment modules can be used to raise or lower the germicidal light emitter 24, such as a retractable cable or the like. For example, cables could be used for vertical distances where a scissor-type lift is impractical, or for cost reasons, or the like. A cable could store and be deployed from a reel (not shown) within a compact bolt-on module (not shown). A motor (not shown) could be used to raise and lower the payload by spooling or releasing cable. The motor could operate the spool via a gear reduction, screw drive mechanism, or the like. An associated rotary encoder (not shown) could allow control of vertical positioning in much the same manner as would be used for a scissor deployment mechanism. If necessary, mechanical stabilization could be added via telescoping or guide rods as deemed appropriate for the specific application. In the example of FIGS. 11 and 12, the z-axis deployment module 22 is connected to an attachment plate 20 that can be fixedly or removably mounted to a bottom of the positioning tractor 16 for movement therewith. The germicidal light emitter 24 is attached to a light mounting plate 52 located opposite the attachment plate 20. FIG. 11 shows the z-axis deployment module 22 in a closed configuration, such as when the UV delivery system 10 is not in use, or is in storage, or where a very tall obstacle is provided on the surface to be disinfected.

FIG. 12 shows the z-axis deployment module 22 in a fully expanded configuration, such as when the surface to be disinfected is located at a maximum height from the positioning tractor 16. Preferably the z-axis deployment module 22 is capable of providing the germicidal light emitter 24 at any vertical position between the closed and fully expanded configurations shown in FIGS. 11-12. In some embodiments, the z-axis deployment module 22 may be in the closed configuration or a substantially closed configuration in order to widen light dispersion (e.g., the coverage footprint). This may lead to a longer exposure time but can greatly increase coverage area.

For example, the position of the germicidal light emitter 24 may be adjusted during operation to conform with changes in the topography of the surface being disinfected. The controller 38 may cause the expansion or retraction of the z-axis deployment module 22. For example, the controller 38 may store a map (e.g., data related to a topography of the surface or vertical distances of points on the at least one surface from the positioning tractor 16). As the positioning tractor 16 moves along the track 12, the controller 38 may adjust the z-axis deployment module 22 according to the map to ensure that the germicidal light emitter 24 is at an appropriate vertical distance from the surface.

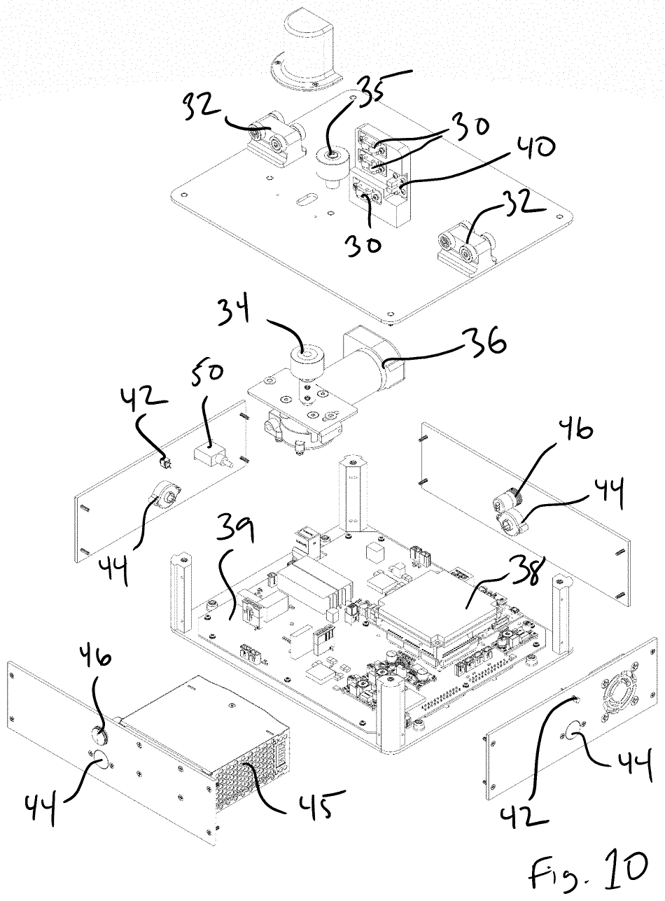

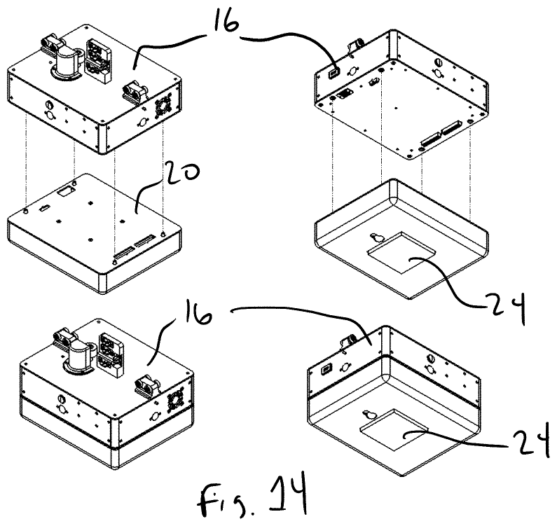

A tilt and/or rotate module 26, such as the example shown in FIG. 13, may be fixedly or removably connected to the light mounting plate 52 or otherwise at the bottom of the z-axis deployment module 24 to allow the germicidal light emitter 24 to be selectively tilted and/or rotated with respect to a longitudinal axis of the z-axis deployment module 24. As shown in FIG. 13, two motors 54, 56 are provided, one controlling tilt and the other controlling rotation for positioning the germicidal light emitter 24 at an optimum angle with respect to the surface. More motors may be used if desired. Operation of the tilt and/or rotate module 26 is preferably controlled by the controller 38. Power and control/data signals can be passed from the positioning tractor 16 to the z-axis deployment module 22, the tilt and/or rotate module 26, and/or other optional components via one or more of power and data ports 58, 60, respectively (see FIG. 9). However, a single port can provide both power and data, or additional ports may be used.

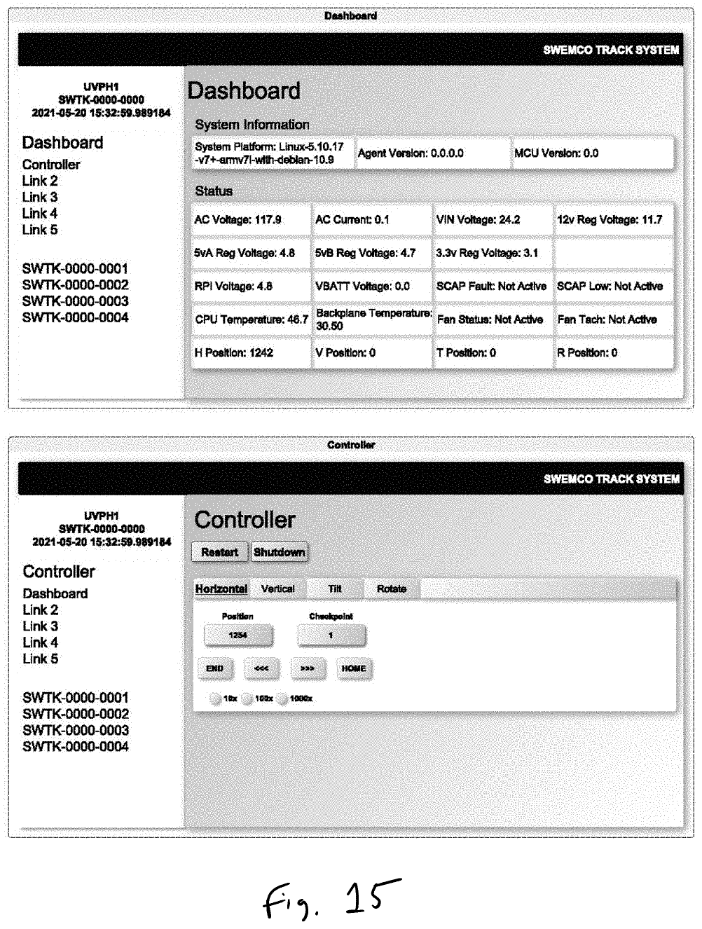

FIG. 14 schematically shows an alternative embodiment of the positioning tractor 16 in which the z-axis deployment module 22 is omitted. The germicidal light emitter 24 can be attached directly to the positioning tractor 16, such as by the attachment plate 20 or the like.

FIG. 15 is a screenshot of an example of a remote portal that may be accessed by a remote or external computer, smartphone, tablet, or other computing device. For example, the remote portal may report status conditions to an operator based on feedback from the various sensors located in the UV delivery system and/or from the onboard controller. For example, the remote portal may report, for example, power state, temperature, a position of the positioning tractor on the track, a position of the z-axis deployment module, a tilt and/or rotation position of the germicidal light emitter, software versions, current location, occupancy sensor status, fault alerts, light status, and the like. The remote portal may also be used to receive command or other data inputs from an operator and send those commands and/or data to the controller in the positioning tractor. Such commands and/or data may include, for example, on/off commands, requests for status updates, commands to change the position of the positioning tractor on the track, the position of the z-axis deployment module, and/or the tilt and/or rotation position of the germicidal light emitter, new map data, firmware updates, and the like. The remote portal may be accessed over a wide area network, such as the Internet or the like, or may be accessible over a local area network, a dedicated communication network, or the like. For example, the remote portal may be accessible through a web browser, an installed software application, or the like.

A prototype of the UV delivery system was built with a Puro pulsed xenon UV germicidal lamp attached and operated. The attached lamp kills up to 99.9% of bacteria and viruses (including Covid-19) at a distance of 10 feet. The prototype implementation was verified capable of attached lamps or lamp arrays drawing up to 10 Amps. This is significantly more than needed to support currently available lamp technologies and multiple lamp arrays.

While the UV delivery system has been described herein as being utilized for disinfecting surfaces and the surrounding air, the positioning tractor and track may be used in conjunction with other equipment for other tasks requiring movement, such as, but not limited to, camera surveillance, food distribution or warehouse product/item movement, and the like.

FIG. 16 shows an example of a more generalized overhead delivery system 100. The example overhead delivery system 100 may include a track 112 attached to a ceiling or other overhead structure (not shown) via track mounting hardware 114. The track 112 and mounting hardware 114 may be similar to those described above for the UV delivery system 10. The track 112 supports a positioning tractor 116 operated to move along the track 112 via a drive system 118. The positioning tractor 116 and drive system 118 may also be similar to those described above for the UV delivery system 10. An underside of the positioning tractor 116 may include an attachment plate 120 supporting a z-axis deployment module 122 for raising and lowering a delivery device 123. The positioning tractor 116, attachment plate 120, and z-axis deployment module 122 may also be similar to those described above for the UV delivery system 10. The delivery device 123 may be oriented using a tilt and rotate module (not shown) similar to that described above with respect to the UV delivery system 10. Power to the overhead delivery system 100 can be at least partially provided by a power input block 128 located at an end of the track 112, similar to that described above with respect to the UV delivery system 10.

In the case of the UV delivery system 10, the delivery device is the germicidal light emitter 24. However, more generally, the delivery device 123 may be, for example, a surveillance camera, a container (e.g., a basket, tote, bin, or the like) configured to hold or deliver food, items, materials, or the like, a claw assembly or similar grasping device configured to hold or deliver food, items, materials, or the like, combinations thereof, or the like. In addition, the delivery device 123 may be or include attachment mechanisms for securing (in some cases removably) devices such as a camera, container, or the like. For example, the delivery device shown in FIG. 16 is a rimmed retainer plate configured to removably receive a material handling tote 125. The z-axis deployment module 122 may be used to descend the delivery device 123 down to receive a filled tote 125 that can be elevated and moved to another location along the track 112 where the tote 125 can be emptied, either automatically or by an individual. In another example, the delivery device 123 may be a hook configured to engage a mating feature on a container, wherein the hook may be descended using the z-axis deployment module 122, engage the container, elevate the container using the z-axis deployment module 122, and deliver the container to another location along the track 112 where the container can then be descended and disengaged from the hook. The generalized overhead delivery system 100 may be suitable for use in moving a device, item, material, or the like between positions during operation.

In yet another embodiment, the system may permit interchangeable delivery devices. For example, the retainer plate 123 may be removed from the z-axis deployment module 122 and replaced with a germicidal emitter light such as the one shown in the earlier drawings. In this way, the system may be used in multiple modes. In furtherance of the previous example, the system may be used during the day to move material and used at night to disinfect the facility. Other combinations may be utilized as well in keeping with the invention.

While specific and distinct embodiments have been shown in the drawings, various individual elements or combinations of elements from the different embodiments may be combined with one another while in keeping with the spirit and scope of the invention. Thus, an individual feature described herein only with respect to one embodiment should not be construed as being incompatible with other embodiments described herein or otherwise encompassed by the invention.

Those skilled in the art will recognize that boundaries between the above-described operations are merely illustrative. The multiple operations may be combined into a single operation, a single operation may be distributed in additional operations and operations may be executed at least partially overlapping in time. Further, alternative embodiments may include multiple instances of a particular operation, and the order of operations may be altered in various other embodiments.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A ceiling or overhead mounted delivery system comprising:
a track configured to be mounted to a ceiling or overhead structure and extend in at least one of a first direction and a second direction, the first direction being substantially orthogonal to the second direction;
a positioning tractor attached to the track for motorized movement thereon;
a selectively actuable germicidal light emitter;
a third direction deployment module coupled to the positioning tractor and the germicidal light emitter and being selectively adjustable to change a distance between the positioning tractor and the germicidal light emitter in a third direction, the third direction being substantially orthogonal to the first and second directions; and
a controller configured to move the positioning tractor along the track, to selectively operate the third direction deployment module to adjust the distance between the positioning tractor and the germicidal light emitter, and to selectively actuate the germicidal light emitter.

2. The system of claim 1, wherein the track includes at least one set of electrical power rails.

3. The system of claim 2, wherein the at least one set of electrical power rails is connectable to one of an alternating current (AC) power supply or a battery.

4. The system of claim 2, wherein the positioning tractor includes at least one electrical contact roller configured for coupling to each respective electrical power rail of the set of electrical power rails.

5. The system of claim 1, further comprising one or more sensors configured to record system information, the system information including at least one of a position of the positioning tractor on the track, a position of the third direction deployment module, a tilt position of the germicidal light emitter, or a rotation position of the germicidal light emitter.

6. The system of claim 5 further comprising a communication module, the controller being configured to report the system information to an external device using the communication module.

7. The system of claim 1, wherein the third direction deployment module is one of a scissor lift or a retractable cable.

8. The system of claim 1, wherein the third direction deployment module includes a tilt and rotate module attached thereto and configured to selectively tilt and rotate the germicidal light emitter.

9. The system of claim 1, wherein the germicidal light emitter is a lamp that emits light in one or more UVC wavelengths.

10. The system of claim 1, wherein the positioning tractor includes at least one occupant sensor, and wherein the controller is configured to shut off the germicidal emitter light based on a signal from the at least one occupant sensor.

11. The system of claim 1, wherein the positioning tractor includes a drive motor and a drive roller operably connected to the drive motor and in contact with the track.

12. The system of claim 1, wherein the controller is disposed within the positioning tractor.

13. The system of claim 1, wherein the track includes a first portion extending in the first direction and a second portion extending in the second direction, and wherein the track further includes a motorized turntable located at a junction between the first and second portions, the motorized turntable being configured to receive the positioning tractor and selectively rotate so as to switch the positioning tractor from the first portion to the second portion of the track.

14. The system of claim 1, further comprising a communication module configured to receive commands from a remote device to change at least one of a position of the positioning tractor on the track, a position of the third direction deployment module, a tilt position of the germicidal light emitter, or a rotation position of the germicidal light emitter.

15. The system of claim 1, wherein the controller is configured to store data related to a topography of a surface to be disinfected by the germicidal light emitter.

16. A vehicle comprising:
a ceiling;
at least one surface to be disinfected located vertically beneath the ceiling;
a power supply;
a track attached to the ceiling;
a positioning tractor attached to the track for motorized movement thereon, the positioning tractor receiving power from the power supply;
a selectively actuable germicidal light emitter;
a vertical deployment module coupled to the positioning tractor and the germicidal light emitter and being selectively adjustable to change a vertical distance between the positioning tractor and the germicidal light emitter; and
a controller configured to move the positioning tractor along the track, to selectively operate the vertical deployment module to adjust the vertical distance between the positioning tractor and the germicidal light emitter, and to selectively actuate the germicidal light emitter to disinfect the at least one surface.

17. The vehicle of claim 16, wherein the power supply is a vehicle battery.

18. The vehicle of claim 16, wherein the at least one surface to be disinfected includes at least one vehicle seat.

19. The vehicle of claim 16, wherein the controller is configured to store data related to vertical distances of points on the at least one surface from the positioning tractor.
